(19) United States
(12) Patent Application Publication
(10) Pub. No.: US 2025/0367457 A1
(43) Pub. Date: Dec. 4, 2025

(54) NEUROSTIMULATION TESTING WITH MINIMAL DISCREET FEEDBACK

(71) Applicant: Boston Scientific Neuromodulation Corporation, Valencia, CA (US)

(72) Inventors: Lisa Denise Moore, Glendale, CA (US); G. Karl Steinke, Valencia, CA (US); Chirag Shah, Valencia, CA (US)

(21) Appl. No.: 19/211,646

(22) Filed: May 19, 2025

Related U.S. Application Data
(60) Provisional application No. 63/652,539, filed on May 28, 2024.

(51) Int. Cl. A61N 1/372; A61N 1/36

(52) U.S. Cl. CPC A61N 1/37264; A61N 1/36132; A61N 1/37241; A61N 1/37247

(57) ABSTRACT

A system may include a neurostimulator, a feedback receiver, and a processing system. The neurostimulator may use program(s) to deliver a neurostimulation therapy to treat a patient condition and provide therapy outcomes. The feedback receiver may receive user feedback about the neurostimulation therapy, the patient condition or the therapy outcomes. The feedback receiver may include sensor(s) to sense user taps on or near the feedback receiver, a swiping motion across at least some of the feedback receiver, and/or feedback receiver movement. The processing system may test stimulation parameter sets including configure the neurostimulator to deliver the electrical energy using each of the tested stimulation parameter sets and use the user feedback to evaluate the tested stimulation parameter sets. The feedback receiver enables the patient to discreetly provide user feedback in a timely manner for a monitoring/programming application.

### CLAIM OF PRIORITY

This application claims the benefit of U.S. Provisional Application No. 63/652,539, filed on May 28, 2024, which is hereby incorporated by reference in its entirety.

### TECHNICAL FIELD

This document relates generally to medical devices, and more particularly, to systems, devices and methods programming a neurostimulation system using user feedback.

### BACKGROUND

Medical devices may include therapy-delivery devices configured to deliver a therapy to a patient and/or monitors configured to monitor a patient condition via user input and/or sensor(s). Examples include wearable devices such as but not limited to, transcutaneous electrical neural stimulators (TENS), external or implantable stimulation devices such as but not limited to spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, and the like.

A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. Settings of the therapy device, including stimulation parameters, may be programmed based on observed clinical effects so that the therapy provides desirable intended effects (e.g., reduced tremor, bradykinesia, and dyskinesia for a PD therapy, desirable pain relief or paresthesia coverage for a pain therapy) while avoiding undesirable side effects.

Programming a neurostimulation adjustment is an ongoing challenge. An in-clinic problem includes the significant time and energy required to test and evaluate programs, as testing and evaluating involves a significant back and forth between a programmer and patient. Another in-clinic problem is that multiple programs may look relatively good in clinic but the at-home outcomes may substantially vary. This disclosure provides an improved system and process for programming the therapy device.

### SUMMARY

An example (e.g., “Example 1”) of a system may include a neurostimulator, a feedback receiver, and a processing system. The neurostimulator may use program(s) to deliver a neurostimulation therapy to treat a patient condition and provide therapy outcomes. The feedback receiver may receive user feedback about the neurostimulation therapy, the patient condition or the therapy outcomes. The feedback receiver may include sensor(s) to sense user taps on or near the feedback receiver, a swiping motion across at least some of the feedback receiver, and/or feedback receiver movement. The processing system may test stimulation parameter sets including configure the neurostimulator to deliver electrical energy using each of the tested stimulation parameter sets and use the user feedback to evaluate the tested stimulation parameter sets. A patient may use the feedback receiver to discreetly and intentionally provide user feedback via taps, swiping motions and/or feedback receiver movement in a timely manner for a monitoring/programming application. The neurostimulator may be an implantable or external neurostimulator. The feedback receiver may be incorporated into one or more devices. For example, the feedback receiver may be incorporated into the neurostimulator, may be incorporated into the processing system, and/or may be incorporated in a device distinct from both the neurostimulator and the processing system. The feedback receiver may send sensor data to the processing system used to analyze the data, or may analyze the sensor data to determine a feedback message and send feedback message to the processing system. The processing system may include one device or the processing system may be distributed across more than one device.

In Example 2, the subject matter of Example 1 may optionally be configured such that the feedback receiver includes at least one of a touch sensor, an accelerometer, acoustic sensor, pressure sensor for sensing at least one of user taps on the feedback receiver, motion of the feedback receiver or swipes across at least a portion of the feedback receiver.

In Example 3, the subject matter of any one or more of Examples 1-2 may optionally be configured to further include at least one user device configured to implement at least a portion of the feedback receiver.

In Example 4, the subject matter of Example 3 may optionally be configured such that the at least one user device includes a body-worn device, and the body-worn device includes the at least one sensor configured to sense the at least one of the user taps, the swiping motion, or the feedback receiver movement.

In Example 5, the subject matter of any one or more of Examples 3-4 may optionally be configured such that the at least one user device includes a phone, a tablet or a remote control.

In Example 6, the subject matter of any one or more of Examples 1-5 may optionally be configured such that the feedback receiver includes at least one sensor in the neurostimulator, and the at least one sensor is configured to sense the user taps.

In Example 7, the subject matter of any one or more of Examples 1-6 may optionally be configured such that the feedback receiver or the processing system is configured to determine the user feedback by detecting a pattern of the at least one of the user taps, the swiping motion, or the feedback receiver movement.

In Example 8, the subject matter of any one or more of Examples 1-7 may optionally be configured such that the feedback receiver or the processing system is configured to determine a user-requested change in the neurostimulation therapy, user acceptance or rejection of the neurostimulation therapy, or user scoring of the neurostimulation therapy by detecting the at least one of the user taps, the swiping motion, or the feedback receiver movement.

In Example 9, the subject matter of any one or more of Examples 1-2 may optionally be configured to further include a wrist-worn device and at least one user device configured to communicate with the neurostimulator and the wrist-worn device, wherein the feedback receiver includes the wrist-worn device and the processing system includes the at least one user device.

In Example 10, the subject matter of Example 1 may optionally be configured such that the feedback receiver includes at least one sensor in the neurostimulator, in a user device, or in a wearable device. Examples of a wearable device include, but are not limited to, a smart watch and smart glasses. Examples of a user device include, but are not limited to, a phone, a tablet or a remote.

In Example 11, the subject matter of any one or more of Examples 1-10 may optionally be configured such that the feedback receiver is configured to produce a user perceivable signal to prompt the user to provide the user feedback. The user perceivable signal may include a vibration and/or a sound.

In Example 12, the subject matter of Example 11 may optionally be configured such that the user perceivable signal is triggered by an occurring or upcoming change in the neurostimulation therapy, the patient condition or the therapy outcomes.

In Example 13, the subject matter of Example 11 may optionally be configured such that the user perceivable signal prompts for a response to a question displayed on a screen.

In Example 14, the subject matter of any one or more of Examples 1-13 may optionally be configured such that the feedback receiver is configured to collect patient feedback in an unprompted manner for patient driven monitoring and reprogramming.

In Example 15, the subject matter of any one or more of Examples 1-14 may optionally be configured such that the processing system is configured to use the user feedback to decide to change stimulation in conjunction with an optimization algorithm, trigger a change in automated patient monitoring from the system, monitor patient outcomes and tag related stimulation data over time, trigger an optimization algorithm to begin running, and/or act as a trigger to change stimulation based on predefined logic.

Example 16 includes subject matter (such as a method, means for performing acts, machine readable medium including instructions that when performed by a machine cause the machine to perform acts, or an apparatus to perform). The subject matter may include using a neurostimulator configured to use at least one neurostimulation program to deliver a neurostimulation therapy to treat a patient condition and provide therapy outcomes, and using a feedback receiver configure to receive user feedback about the neurostimulation therapy, the patient condition or the therapy outcomes. Receiving the user feedback may include using at least one sensor to sense at least one of user taps on or near the feedback receiver, a swiping motion across at least some of the feedback receiver, or feedback receiver movement. The subject matter may further include testing stimulation parameter sets using a processing system, including configuring the neurostimulator to deliver electrical energy using each of the tested stimulation parameter sets and using the user feedback to evaluate the tested stimulation parameter sets.

In Example 17, the subject matter of Example 16 may optionally be configured such that the user feedback is received using at least one of a touch sensor, an accelerometer, acoustic sensor, pressure sensor for sensing at least one of user taps on the feedback receiver, motion of the feedback receiver or swipes across at least a portion of the feedback receiver.

In Example 18, the subject matter of any one or more of Examples 16-17 may optionally be configured such that the user feedback is received using at least one user device.

In Example 19, the subject matter of any one or more of Examples 16-18 may optionally be configured such that the user feedback is received using a body-worn device to sense at least one of user taps, the swiping motion, or the feedback receiver movement.

In Example 20, the subject matter of any one or more of Examples 16-19 may optionally be configured such that the user feedback is received using a phone, a tablet or a remote control.

In Example 21, the subject matter of any one or more of Examples 16-20 may optionally be configured such that the user feedback is received using the neurostimulator to sense the user taps.

In Example 22, the subject matter of any one or more of Examples 16-21 may optionally be configured to further include determining the user feedback by detecting a pattern of the at least one of the user taps, the swiping motion, or the feedback receiver movement using the feedback receiver or the processing system.

In Example 23, the subject matter of any one or more of Examples 16-22 may optionally be configured to further include using the feedback receiver or the processing system to determine a user-requested change in the neurostimulation therapy, user acceptance or rejection of the neurostimulation therapy, or user scoring of the neurostimulation therapy by detecting the at least one of the user taps, the swiping motion, or the feedback receiver movement.

In Example 24, the subject matter of any one or more of Examples 16-23 may optionally be configured such that the feedback receiver includes a wrist-worn device and the processing system includes at least one user device configured to communicate with the wrist-worn device.

In Example 25, the subject matter of any one or more of Examples 16-24 may optionally be configured such that the user feedback is received using at least one sensor in the neurostimulator, in a user device, or in a wearable device.

In Example 26, the subject matter of any one or more of Examples 16-25 may optionally be configured to further include producing a user perceivable signal using the feedback receiver to prompt for the user feedback. The user perceivable signal may include at least one of a vibration, a sound or a visual notification.

In Example 27, the subject matter of Example 26 may optionally be configured to further include triggering the user perceivable signal based on an occurring or upcoming change in the neurostimulation therapy, the patient condition or the therapy outcomes.

In Example 28, the subject matter of any one or more of Examples 26-27 may optionally be configured to further include using the user perceivable signal to prompt for a response to a question displayed on a screen.

In Example 29, the subject matter of any one or more of Examples 16-28 may optionally be configured to further include collecting patient feedback in an unprompted manner using the feedback receiver, and using the collected patient feedback to provide patient driven monitoring of the neurostimulation therapy and reprogramming the neurostimulator.

In Example 30, the subject matter of any one or more of Examples 16-29 may optionally be configured to further include using the user feedback to decide to change stimulation in conjunction with an optimization algorithm, trigger a change in automated patient monitoring, monitor patient outcomes and tag related stimulation data over time, trigger an optimization algorithm to begin running, and/or act as a trigger to change stimulation based on predefined logic.

Example 31 includes subject matter that includes non-transitory machine-readable medium including instructions, which when executed by a machine, cause the machine to perform a method. The method may include, by way of example and not limitation, any of the subject matter for at least portions of one or more of Examples 16-30. By way of example, the method may include using a feedback receiver configured to receive user feedback about a neurostimulation therapy to treat a patient condition and provide therapy outcomes, including using at least one sensor to sense at least one of user taps on or near the feedback receiver, a swiping motion across at least some of the feedback receiver, or feedback receiver movement, and the method may further include testing stimulation parameter sets using a processing system, including configuring a neurostimulator to deliver electrical energy using tested stimulation parameter sets and using the user feedback to evaluate the tested stimulation parameter sets. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system that may be used to deliver deep brain stimulation (DBS).

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device for programming or controlling the operation of an electrostimulation system.

FIG. 5 illustrates an example of a stimulation parameter control system and a part of the environment in which it may operate.

FIG. 6 illustrates, by way of example, an example of an electrical therapy-delivery system.

FIG. 7 illustrates, by way of example and not limitation, an implantable electrical therapy-delivery system.

FIG. 8 illustrates a therapy being delivered according to a parameter set.

FIG. 9 illustrates a therapy space, which includes different parameter sets potentially available for delivering the therapy.

FIG. 10 illustrates, by way of example and not limitation, a system that includes a stimulator, a feedback receiver and a processing system, where the feedback receiver includes sensor(s) for sensing user action feedback.

FIG. 11 illustrates, by way of example and not limitation, a system that includes a stimulator and a processing system, where user device(s) of the processing system includes the feedback receiver with sensor(s) for sensing user action feedback.

FIG. 12 illustrates, by way of example and not limitation, a system that includes a stimulator and a processing system, where the stimulator includes the feedback receiver with sensor(s) for sensing user action feedback.

FIG. 13 illustrates, by way of example and not limitation, examples of sensed user actions that may be used to receive user feedback.

FIG. 14 illustrates, by way of example and not limitation, a processing system which may be used to monitor the neurostimulation therapy, the patient condition or the therapy outcomes and to program the neurostimulator.

FIG. 15 illustrates, by way of example and not limitation, a feedback receiver with a sensor data analyzer configured to analyze the sensor data and communicate feedback to a monitoring and/or programming application in the processing system.

FIG. 16 illustrates, by way of example and not limitation, a feedback receiver and a processing system with a sensor data analyzer configured to analyze the sensor data from the feedback receiver to provide feedback to a monitoring and/or programming application in the processing system.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Disclosed herein, among other things, are systems and methods for programming a medical device when the patient is away from a clinic. By way of examples, a brief schedule may be used to compare outcomes of two or more programs, or an algorithm may be implemented to optimize the programming based on the patient feedback. A programming algorithm may be implemented when the patient is home or when the patient is away from home like at a restaurant, at an entertainment venue, at a social event, or traveling. The systems, devices and methods may enable the patient to discreetly provide user feedback in a timely manner for a monitoring/programming application. The patient feedback is easily used by a large percentage of the patients. The patient feedback involves a minor interaction in many cases when the therapy is well managed while enabling a patient to provide user feedback about the neurostimulation therapy, the patient condition or the therapy outcomes when therapy changes may be warranted. The algorithm may be implemented using a system of devices. A system of devices may include at least one device is used to provide user feedback and other device(s) used to determine the neurostimulation parameters to program or determine a set of questions or prompts for receiving the feedback. The system of devices may include sensor(s) configured to sense physiological effects of the neurostimulation therapy including therapeutic effects and side effects of the stimulation, the patient condition or the therapy outcomes.

DBS is used as a specific example of neurostimulation herein. A DBS system is described in more detail below. The present subject matter may be applied to other therapy systems that may struggle with a lengthy, iterative process to program and evaluate therapies.

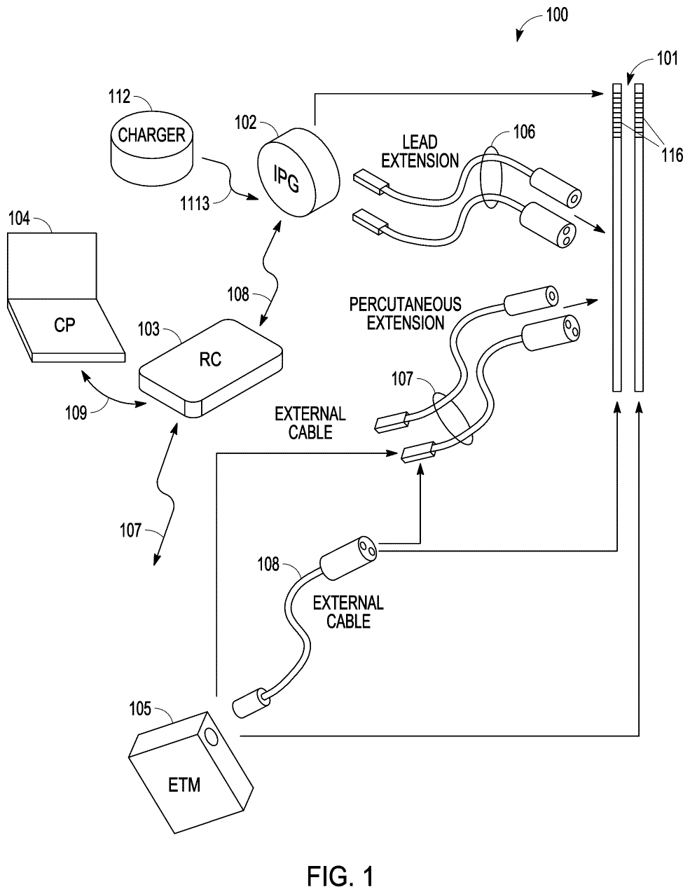

FIG. 1 illustrates, by way of example and not limitation, an electrical stimulation system 100, which may be used to deliver DBS. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

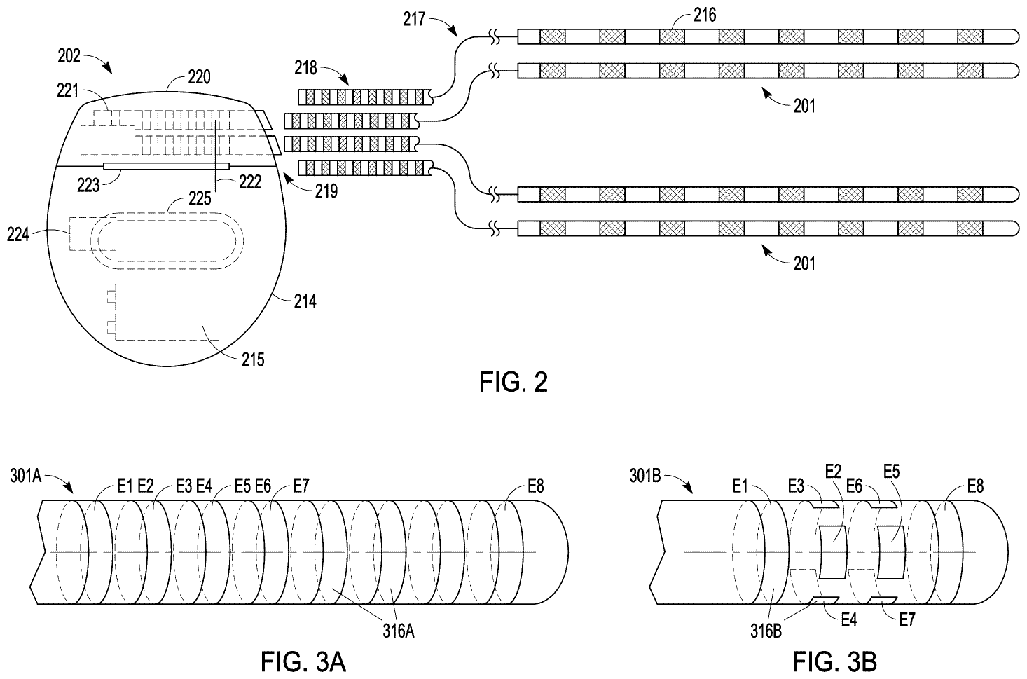

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

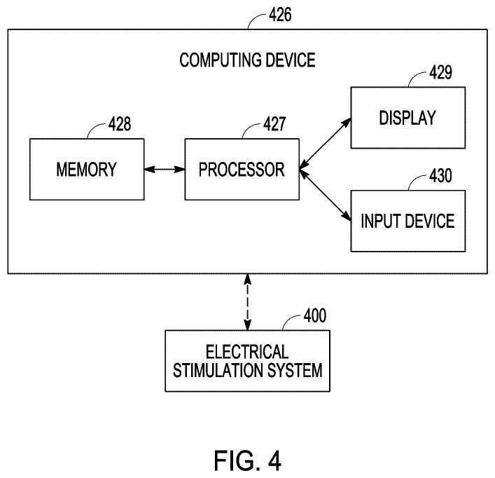

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1.

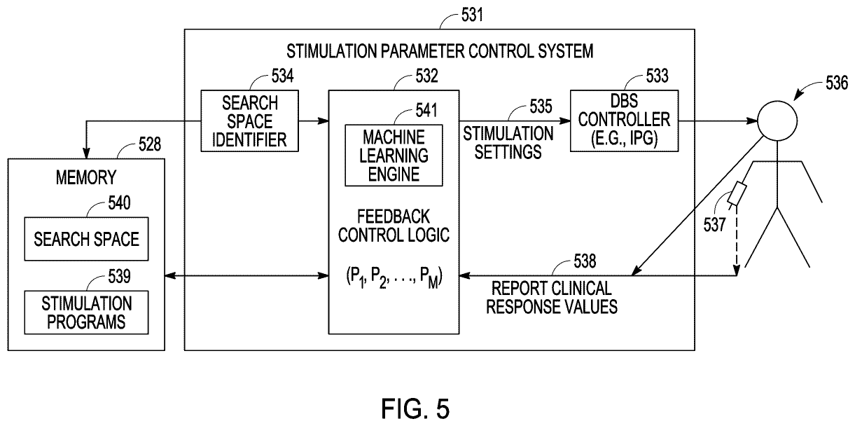

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses, as described below with reference to FIG. 5. Examples of the patient clinical responses may include physiological signals (e.g., heart rate) or motor parameters (e.g., tremor, rigidity, bradykinesia). The computing device 426 may communicate with the axis. CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, the closed-loop algorithm for determining or updating stimulation parameters may be implemented in a mobile device, such as a smartphone, that is connected to the IPG or an evaluating device (e.g., a wrist-worn device such as a wristband or watch). These devices may also record and send information to the clinician.

The processor 427 may include one or more processors that may be local to the user or non-local to the user or other components of the computing device 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A stimulation setting includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like.

The processor 427 may identify or modify a stimulation setting from the search space through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth wireless technology, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

FIG. 5 illustrates, by way of example and not limitation, a stimulation parameter control system and a part of the environment in which it may operate. The stimulation parameter control system 531 may be an example of a monitoring and programming application. The stimulation parameter control system 531, which may be implemented as a part of the processor 427 in FIG. 4, may include a feedback control logic 532, a DBS controller 533, and a search space identifier 534. DBS is used as an example. It is noted that the system may be implemented for other stimulation therapies such as, but not limited to, SCS or PNS. The feedback control logic 532 may be implemented in, for example, the CP 104 or the RC 103 in FIG. 1. The feedback control logic 532 may determine or modify one or more stimulation settings 535 for a stimulation lead at a target stimulation region, such as a region in a brain hemisphere. A stimulation setting may include an electrode configuration and values for one or more stimulation parameters (P₁, P₂, . . . , Pₘ). The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 532 may modify the stimulation setting 535 such as by changing a stimulation parameter value, or modifying an electrode configuration.

The stimulation setting 535 may be provided to the DBS controller 533 to configure the IPG or ETM to deliver DBS therapy to the patient 536 in accordance with the stimulation setting or the modified stimulation setting. The stimulation may produce certain therapeutic effects and/or side effects on the patient 536. Such therapeutic effectiveness and side effects, also referred to as clinical responses or clinical metrics, may be provided to the feedback control logic 532. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 537. In an example, the sensor 537 may be included in a wearable device associated with patient 536, such as a wrist-worn device like a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 538, also referred to as clinical response scores. In an example, the clinical response values 538 may be computed based on the intensity, frequency, or duration of one or more of tremor, rigidity, or bradykinesia responses. Based upon the received clinical response values 538, the feedback control logic 532 may adjust electrode configurations or values of one or more stimulation parameters 535. The feedback control logic 532 may send the adjusted (new or revised) stimulation setting 535, such as the electrode configuration or the adjusted stimulation parameter values, to further configure the DBS controller 533 to change the stimulation parameters of the leads implanted in patient 506 to the adjusted values.

The feedback-control loop as illustrated in FIG. 5 may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values 538 exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). Accordingly, three optimal base stimulation settings may be generated: a first optimal base stimulation setting (BSS₁) corresponding to a bradykinesia score exceeding a threshold, a second optimal base stimulation setting (BSS₂) corresponding to a tremor score exceeding a threshold, and a third optimal base stimulation setting (BSS₃) corresponding to a rigidity score exceeding a threshold. In another example, the clinical response values may be a composite score computed as a weighted combination of multiple clinical effects, such as a %*bradykinesia+b %*tremor+c %*rigidity. Accordingly, a fourth optimal base stimulation setting (BSS₄) may be generated, corresponding to the composite clinical response score exceeding a threshold. In some examples, the stimulation setting optimization may be performed in an in-clinic programming session such during implantation or revision of a DBS system or device follow-up.

The optimal base stimulation settings (e.g., BSS₁ through BSS₄), may be stored in the memory 528. In an example, a stimulation setting, along with the corresponding unique clinical response indicator (e.g., weighted combination of clinical effects with unique weight factors) form a stimulation program 539, which may also be stored in the memory 404. Each stimulation programmed may be associated with, or tagged by, one or more unique clinical response indicators. In some examples, the clinical response values 538 may be weighted according to the time at which the test took place.

In various examples, the stimulation parameter control system 531 may be executed on its own and is not connected to a controller. In such instances it may be used to merely determine and suggest programming parameters, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations and combinations of stimulation parameter values) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings such as BSS₁-BSS₄ as discussed above.

The search space identifier 534 may automatically determine a search space 540 for a stimulation lead at a neural target, such as a region in a brain hemisphere, by imposing certain limitations or constraints on the electrode configurations and/or parameter values or value ranges. In an example, the search space 540 may be determined based on spatial information of the lead, such as lead positions with respect to neural targets, which may be obtained from imaging data of the lead and patient anatomy. Additionally, or alternatively, the search space 540 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 540. In an example, the search space identifier 534 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 532 may determine one or more optimal base stimulation settings (e.g., BSS₁-BSS₄) by searching through the identified search space 540. The identified search space 540 may be stored in the memory 528.

The feedback control logic 532 may include a machine learning engine 541 that may facilitate the stimulation parameter control system 531 (or a user of the system) to explore the search space in order to choose values for programming the DBS controller 533. The machine learning engine 541 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 533 to deliver stimulation accordingly to the patient 536, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 541 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 531. With its learning and prediction capability, the machine learning engine 541 may aid a user (e.g., a clinician) in exploring the stimulation parameter space more effectively and more efficiently to produce results that are optimal, desired, or acceptable.

In some examples, the machine learning engine 541 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 541 may determine expected outcomes for parameter values that have not yet been tested based upon what the machine learning engine 541 has “learned” thus far and provide a recommendation for a next set of values to test. Here, testing refers to the iterative testing required to find an optimal stimulation setting for configuring the DBS controller 533. The recommendation for a next set of values to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 532 with its machine learning engine 541 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 541. In some examples, no prior data may be used. In this case, the machine learning engine 541 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 541 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 541 may decide whether a particular predicted outcome for a set of stimulation parameter values is the fastest sufficing outcome, the best possible clinical outcome, or the optimal outcome with least battery usage, for example.

The feedback control logic 532 may be used to search and configure different types of stimulation parameters of the various leads potentially causing different clinical effects upon the patient 536. Examples of the stimulation parameters may include electrode configurations (electrode selection, polarities, monopolar or bipolar modes of stimulation), current fractionalization, current amplitude, pulse width, frequency, among others. Given these possible stimulation parameters, the stimulation parameter control system 531 may move about the parameter space in different orders, by different increments, and limited to specific ranges. In some examples, the stimulation parameter control system 531 may allow the user (such as a clinician, physician, programmer, etc.) to provide search range limitations to one or more of the stimulation parameters to limit the range for that stimulation parameter over which the system will search for parameters. For example, the user may restrict which electrodes may be used for stimulation or may restrict the amplitude or pulse width to a certain range or with a selected maximum or minimum. As one illustration, based on the site of implantation, the user may be aware that the distal-most and proximal-most electrodes are unlikely to produce suitable stimulation and the user limits the range of electrodes to exclude these two electrodes.

For a lead with segmented electrodes, the number of possibilities for parameter selection may be very large when combinations of electrodes and different amplitudes on each electrode are possible. In some examples using a lead with segmented electrodes, the selection of electrodes used for stimulation may be limited to fully directional selections (i.e., selection of only a single segmented electrode) and fully concentric selections (i.e., all electrodes in a single set of segmented electrodes are active with the same amplitude). In other examples, the initial movement through parameter space may be limited to fully directional and fully concentric selections. After a set of stimulation parameters is identified using these limits, variation in the selection of electrodes may be opened up to other possibilities near the selection in the identified set of stimulation parameters to further optimize the stimulation parameters.

In some examples, the number of stimulation parameters that are varied and the range of those variations may be limited. For example, some stimulation parameters (e.g., electrode selection, amplitude, and pulse width) may have larger effects when varied than other stimulation parameters (e.g., pulse shape or pulse duration). The movement through stimulation parameter space may be limited to those stimulation parameters which exhibit larger effects. In some examples, as the stimulation parameter control system 531 proceeds through testing of sets of stimulation parameters, the system may observe which stimulation parameters provide larger effects when varied and focus on exploring variation in those stimulation parameters.

In some examples, the stimulation parameter control system 531 may include a user interface for visualizing exploration of the stimulation parameter space as the system determines new and better parameter values to test until a solution is determined that fits within certain designated thresholds or a stop condition is reached. In some examples of the stimulation parameter control system 531, the user interface is part of the feedback control logic. In other examples, the user interface may be part of another computing system that is part of the stimulation parameter control system 531 or may be remote and communicatively connected to the stimulation parameter control system 531. The user interface may present to a user (such as a clinician, physician, programmer, etc.) a visualization of the predicted expected outcomes for (some of) the stimulation parameter values not yet tested and a recommendation for the next set of stimulation parameter values to test.

In some examples where a deep brain stimulator is configured via the DBS controller 533 with at least one set of stimulation parameter values forwarded by the feedback control logic 532, the clinician may monitor the patient throughout the process and record clinical observables in addition to the patient 536 being able to report side effects. When a side effect is observed, the various search algorithms may take that fact into account when selecting/suggesting a next set of values to test. In some examples, for example, those that select contacts via monopolar review, other parameters may be changed until they cause a side effect, which case is noted as a boundary. For example, in monopolar review where amplitude is another stimulation parameter being varied, the amplitude may be increased progressively until a side-effect is observed.

In some examples, more than one clinical metric (e.g., tremor, rigidity, bradykinesia, etc.) may be important observables. Different examples of the stimulation parameter control system 531 may handle these metrics differently. For example, some examples might identify an ideal location for each metric and choose one ideal location between them, set in the patient's remote controller so the patient may choose as needed, or chose a best combined outcome. As another example, some examples may search multiple outcomes at the same time and use the best combined score as the best outcome or find a best location for each metric individually. As yet another example, some examples may use a sequential process for selecting stimulation parameter values for multiple outcomes. For example, a system may search parameter space for a first outcome (e.g., bradykinesia) and, upon finding a suitable end condition, then search parameter space for a second outcome (e.g., rigidity). While searching parameter space for the first outcome, clinical response values for both the first and second outcomes may be obtained. Thus, when the system switches to the second outcome there are already a number of clinical response values for that outcome which will likely reduce the length of the search.

In some examples, two stimulation leads may be implanted to produce stimulation effects on two sides of the body (e.g., the right and left sides of the body). The same procedure described herein may be used to either jointly determine the stimulation parameters for the two leads by exploring the joint parameter space or individually determine stimulation parameters for the two leads by exploring the parameter space for each lead individually. In some examples, the user may determine for each side of the body which clinical response is dominant or most responsive. This may be done, for example, by having the patient perform a single task which captures multiple responses (e.g., connecting dots on the screen to monitor tremor and bradykinesia of the movement) or a small series of tasks. This enables the system to determine which clinical response to use to identify the stimulation parameters for that side of the body.

As noted, the feedback may be provided directly by the patient 536, entered by an observer such as a clinician (not shown), or may be provided by means of a sensor 537 associated with and in physical, auditory, or visual contact with the patient 536. Examples may include, but are not limited to, accelerometers, microphones, and cameras. In an example, the sensor 537 may be included in a wearable device associated with patient 536, such as a smart watch. In an example where the feedback may be monitored automatically or semi-automatically, such as with use of sensor 537, it may not be necessary for a clinician or other observer to be present to operate the stimulation parameter control system 531. Accordingly, in such examples a user interface may not be present in system 531.

In some examples, the stimulation parameter control system 531 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings (e.g., BSS₁-BSS₄) that are based on the tested clinical response (either reported by the patient or measured by a sensor) to actually delivered stimulation. For examples, based on the “tested” base stimulation settings BSS₁-BSS₄, the stimulation parameter control system 531 may estimate an optimal base stimulation setting associated with a composite clinical response defined as x %*bradykinesia+y %*tremor+z %*rigidity, or simply denoted by the weight factors (x %, y %, z %). By way of example and not limitation, the stimulation parameter control system 531 may generate a fifth optimal base stimulation setting (BSS₅) corresponding to a composite clinical response using bradykinesia and tremor only, each weighted 50%; a sixth optimal base stimulation setting (BSS₆) corresponding to a composite clinical response using tremor and rigidity only, each weighted 50%; a seventh optimal base stimulation setting (BSS₇) corresponding to a composite clinical response using bradykinesia and rigidity only, each weighted 50%; or an eighth optimal base stimulation setting (BSS₈) corresponding to a composite clinical response using bradykinesia, tremor, and rigidity weighted 40%, 40%, and 20%, respectively. Similar to the tested base stimulation settings BSS₁-BSS₄, the estimated base stimulation settings BSS₅-BSS₈, associated with their respective clinical response indicators (e.g., weight factors for clinical effects), may be stored in the memory 528 as respective stimulation programs 539. In an example, the stimulation programs 539 may be stored in a lookup table, where each tested or estimated base stimulation setting (e.g., BSS₁ through BSS₈) may be tagged by respective clinical response indicators or weight factors for clinical effects. In an example, the memory 528 may be a part of memory circuitry internal to the IPG. The RC or the CP may request access to the memory 528 to retrieve therefrom one or more stored stimulation programs 539 or the search space 540.

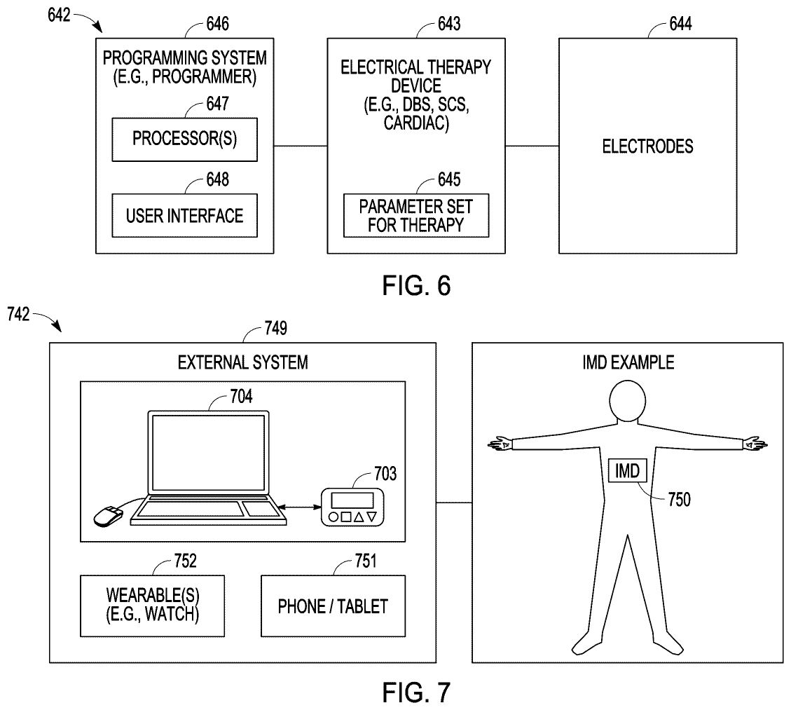

FIG. 6 illustrates, by way of example, an example of an electrical therapy-delivery system. The illustrated system 642 includes an electrical therapy device 643 configured to deliver an electrical therapy to electrodes 644 to treat a condition in accordance with a programmed parameter set 645 for the therapy. The system 642 may include a processing system 646 that may include one or more processors 647 and a user interface 648, which may be used to program and/or evaluate the parameter set(s) used to deliver the therapy. The illustrated system 642 may be a DBS system for treating a movement disorder, such as has been illustrated and discussed with respect to FIGS. 1-5, and/or a system for monitoring the movement disorder.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

FIG. 7 illustrates, by way of example and not limitation, the electrical therapy-delivery system of FIG. 6 implemented using an implantable medical device (IMD). The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off and/or may allow the patient to adjust patient-programmable parameter(s) of the plurality of stimulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The phone and/or tablet may include camera(s), microphone(s), accelerometer(s) or other sensors that can be used to provide feedback. The system may include physical sensors. In nonlimiting examples, the physical sensor may be configured for use in detecting or determining rigidity, stiffness, muscle tension, or movement. The physical sensors may include internal physical sensors or external physical sensors. The system 742 may include medical record(s) for the patient and broader patient population(s). The medical record(s) may be stored and accessed using one or more servers (e.g., local or remote servers such as cloud-based servers). The external device may also include device(s) (e.g., app on phone/tablet or a custom device) used by the patient to perform tasks and may also monitor the ability of the patient to perform the task. The external system may be used to process inputs, detect events, analyze the results and/or optimize the training. Processing may be done using cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

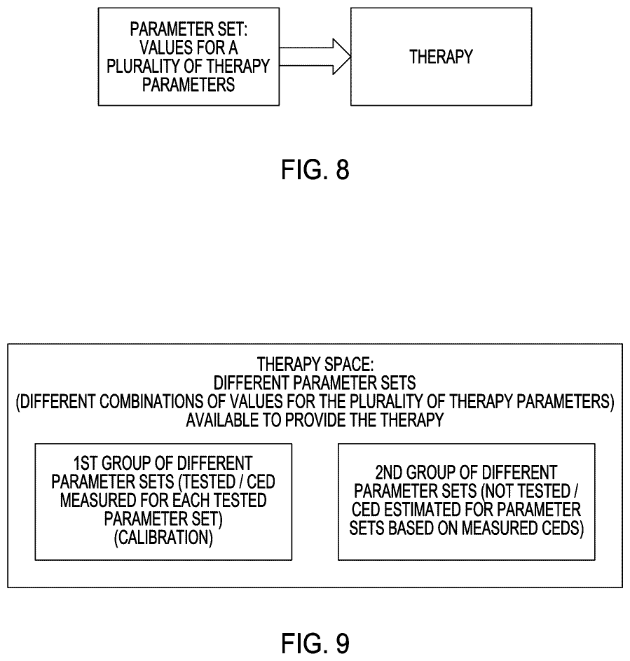

FIG. 8 illustrates a therapy being delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters.

FIG. 9 illustrates a therapy space, which includes different parameter sets potentially available for delivering the therapy. The different parameter sets have unique combinations of values for the therapy parameters. The therapy space may be burdensomely large as there may be many unique combinations of values for therapy parameters (e.g., many unique parameter sets). Some parameter sets within the therapy space may be tested and the corresponding clinical effect data (CED) may be measured or otherwise acquired for the tested parameter sets. These tested parameter sets are illustrated as a first group of different parameter sets within the therapy space. Other parameter sets may not be tested (e.g., second group of different parameter sets). The CED for these parameter sets may be estimated based on measured CEDs for the patient or a patient population.

For example, CED may be directly measured to provide calibration settings. The therapy sessions may be delivered using different therapy settings, and the CED may be recorded for each session. For a neurostimulator such as DBS, SCS, PNS, TENS or FES, for example, the therapy may involve delivering electrical waveforms, which may be a pulsed waveform. Programmable settings for the pulse waveform may include a pulse amplitude, a pulse width, a pulse frequency, a pulse train duration, a pulse-to-pulse duty cycle, a pulse train to pulse train duty cycle (stimulation ON/OFF), and a stimulation schedule (e.g., programmable start and/or stop times, such as but not necessarily a calendar-based schedule). The programmable settings may further include controlling which of a plurality of electrodes are active and which are off, the polarity of each active electrode (which active electrode(s) are anode(s) and which are cathode(s), and the contributions (e.g., electrode fractionalization) of total energy delivered to individual one(s) of the anode(s) and individual one(s) of the cathode(s). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. Controlling the individual contributions by individual electrodes adjusts the location and shape of the stimulation field, to modulate different combinations of neural elements. The settings may be spread throughout the stimulation space for use in identifying clinical responses from session to session, including both the previously-measured clinical effects for tested stimulation settings and predicted or estimated clinical effects for stimulation settings that were not previously tested.

The responses or stimulation effects for tested parameter sets may include patient responses associated with patient outcomes which may be referred to as clinical effects data. Patient outcomes may include perception, therapeutic effects, and side effects. The responses or stimulation effects for tested parameter sets may also include sensed data.

Given the large number of available parameters sets, it is not practical to test all possible parameter sets to map the corresponding stimulation effects for the parameter sets. However, based on what has been tested, stimulation effect predictions (e.g., estimated responses) may be made for parameter sets that were not tested. By way of example and not limitation, these predictions may be used to determine a good parameter set for testing or for therapy.

Disclosed herein are improved systems, devices and methods for programming a neurostimulation system using user feedback. For example, a system may include a neurostimulator, such as an implantable pulse generator or an external pulse generator, and a feedback receiver capable of receiving input such as tap, motion or swipe-based feedback. A paired mobile application may coordinate programming and evaluation processes for the at-home patient. For example, a mobile device implementing the mobile application may know the status of stimulation and may modulate stimulation in response to data collected from the feedback receiver which may collect patient feedback in a prompted manner for decision making timepoints and/or may collect patient feedback in an unprompted manner for patient driven monitoring/reprogramming. The feedback receiver is easily used by a large percentage of the patients, and can accommodate situations where the patient is doing well and does not need or want intervention and can also accommodate situations where additional user feedback is warranted to improve the therapy. The systems and methods may enable the patient to discreetly provide user feedback in a timely manner for a monitoring and/or programming application.

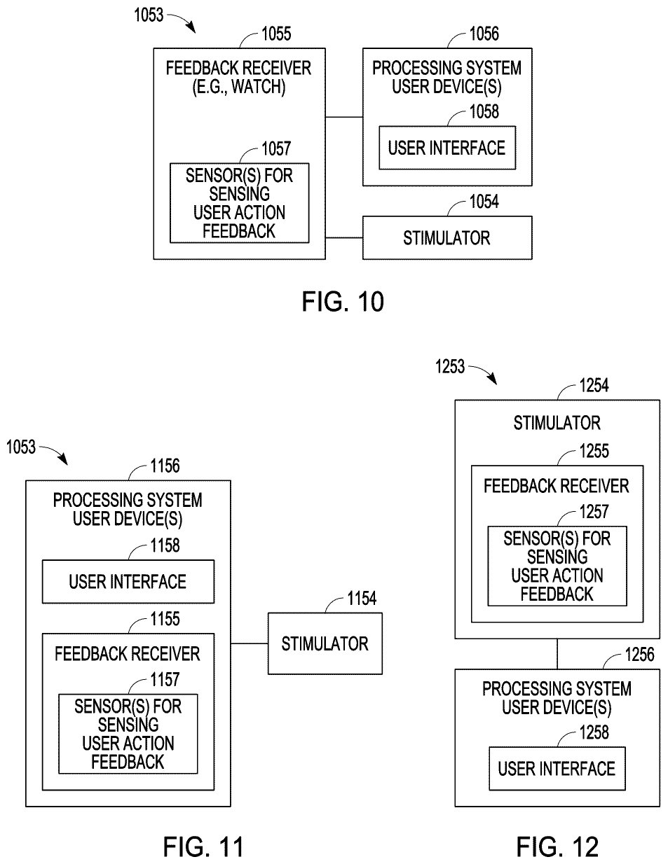

FIG. 10 illustrates, by way of example and not limitation, a system 1053 that includes a stimulator 1054, a feedback receiver 1055 and a processing system 1056, where the feedback receiver 1055 includes sensor(s) 1057 for sensing user action feedback. The processing system 1056 may include one or more user devices such as a phone, tablet, wearable device, or remote control, where at least some of the device(s) include a user interface 1058. The user action feedback is an intentional, albeit discreet, interaction by the patient with the system to communicate feedback information to the system. The discreet interaction may be only a minor inconvenience for the patient and may often be performed unnoticed by others in the patient's vicinity. The communicated feedback information may correspond to the patient-provided responses that may be converted to clinical response values 538 in FIG. 5. The feedback receiver 1055 may be its own device distinct from user device(s) in the processing system 1056 and distinct from the stimulator 1054. At least a portion of the feedback receiver 1055 may incorporated in a device with the stimulator 1054 or the processing system 1056. For example, an app implemented a user device may implement functions associated with at least a portion of the feedback receiver 1055 and another app implemented the user device may implement functions associated the processing system 1056. Different processes or threads may be used to implement the different apps.

Various embodiments include a wearable device or devices, such as but not limited to a smart watch, smart band, other smart body-worn devices such as smart glasses, or other smart carried or handheld devices, for a patient to provide feedback the neurostimulation therapy, the patient condition or the therapy outcomes. The wearable may function as a tap receiver to provide a patient driven feedback mechanism that may be used to report stimulation outcomes. Taps are one example of intentional user actions that can be detected by the sensor(s). Other types of feedback include simple taps, motions, swipes or a combination or pattern of one or more of these to indicate the patient's perception of their therapy outcomes from the neurostimulation. The feedback may include more complicated combinations of user actions. This may be used alone or in combination with an app that can prompt additional questions. The sensor(s) may sense a patient's touch. Examples of such technology may include pressure sensors, acoustic sensor(s) like a microphone, temperature sensors, accelerometers, and capacitive or resistive touch screen technology. The sensor(s) may sense motion. An example of a motion sensor includes an accelerometer. The feedback may include button presses, physical toggles, and the like.

Patterns of feedback can quickly indicate the patient's perception of their therapy. For example, the patient may signal, via a tap or tap pattern, that the therapy outcomes are satisfactory and may only provide the signal when prompted to do so. A pattern of such responses may indicate that the program is acceptable or nearly acceptable. In another example, a patient may quickly provide an unprompted response to indicate that the stimulation therapy is uncomfortable. The unprompted response may trigger additional queries for receiving additional feedback either using the feedback receiver 1055 or a user device in the processing system 1056. A pattern of such responses may suggest that the parameters should be significantly changed (e.g., lowered) to avoid the discomfort. The tap-based feedback on a wearable or other patient feedback device may be used to record neurostimulation (e.g., DBS) monitoring outcomes. The tap-based feedback on the wearable or other patient feedback device may be used to change stimulation. The tap-based feedback may be used on a wearable to cycle through, approve or alter schedules or recommend programming changes.

Various embodiments provide a feedback receiver 1055 configured to prompt the patient to provide feedback the neurostimulation therapy, the patient condition or the therapy outcomes. For example, a feedback receiver 1055 may provide a user perceivable signal, such as but not limited to a vibration or sound, to indicate a request for feedback. The user perceivable signal may be triggered by an occurring or upcoming change in the neurostimulation therapy, the patient condition or the therapy outcomes. The feedback receiver 1055 may include a phone or a wrist-worn device such as smart watch. The signal pattern, such as vibration, noise emitted, visual notification, and the like, may be used to indicate what feedback is being requested. A patient's response to the prompt may be simple taps, motions, swipes or a combination or pattern of these. An example of a system may use a wearable device (e.g., watch) to communicate a recommended stimulation change as part of an automated remote programming workflow. The wearable may vibrate, emit noise, or otherwise indicate a new stimulation suggestion. The user may use simple taps, motions, swipes or a combination or pattern of these actions to indicate that they wish to accept/reject/skip the recommendation or to stop the automated remote programming process. The feedback may include more complicated combinations of user combinations. The feedback may include button presses, physical toggles, and the like.

The system may be designed to provide a variety of patient-provided feedback items through the feedback receiver 1055. The feedback receiver 1055 may allow a patient to provide feedback or may serve as a simplified remote to control stimulation. The feedback only configuration for the system may communicate: On time (symptoms being well managed without troublesome side effects) or Off time; the presence and/or severity of symptoms; the presence and/or severity of side effects, the patient's satisfaction with the therapy; and an ability to accomplish a goal; the perception of pain, or taking medication. The input may serve to mark a time, activity, program or elements thereof as being acceptable, desirable, unacceptable, undesirable, improved, worsened, preferred, or the like, or various combinations thereof. The system configuration for requesting a change to stimulation may include a request to skip a program that is part of a schedule or optimization algorithm, a patient's acceptance or rejection of a stimulation change, user scoring of the neurostimulation therapy, a change in a program, an increase or decrease in amplitude, or a turn stimulation ON or stimulation OFF processing system includes the feedback receiver with sensor(s) for sensing user action feedback. Prompted and unprompted patient feedback may be used to decide to change stimulation in conjunction with an optimization algorithm, trigger an increase in automated patient monitoring from the system which could increase the prompted responses and change the data requested, monitor patient outcomes and tag related stimulation data over time as a method of understanding outcomes, trigger an optimization algorithm to begin running, and/or act as a trigger to change stimulation based on predefined logic. A benefit of using the present subject matter for automated programming includes reducing or minimizing the burden and increasing or maximizing the outcomes data.

The system may allow the stimulator to execute algorithms that can optimize a patient's stimulation at home. The wearable device, when implemented as a feedback receiver, may be used as a communication tool that is highly adopted and does not rely on the patient having their mobile phone or RC with them. Simple patterns of vibration or noise from the wearable can communicate a variety of simple feedback requests from the patient. Simple pattern of taps, motions, swipes on the wearable from the patient can indicate a response to a request or can be used to provide unprompted feedback. Furthermore, the system may create a simple communication means between the system and the patient that can be executed subtly in public without the patient feeling like they are obviously interacting with a medical device.

FIG. 11 illustrates, by way of example and not limitation, a system 1153 that includes a stimulator 1154 and a processing system 1156, where user device(s) of the processing system 1156 includes the feedback receiver 1155 with sensor(s) for sensing user action feedback. The sensor(s) may sense a patient's touch. Examples of such technology may include pressure sensors, acoustic sensor(s) like a microphone, temperature sensors, accelerometers, and capacitive or resistive touch screen technology. The sensor(s) may sense motion. An example of a motion sensor includes an accelerometer. The processing system 1156 may include one or more user devices such as a phone, tablet, wearable device, or remote control, where at least some of the device(s) include a user interface 1158. The processing system 1156 may further include data centers or servers. Thus, the phone may function to provide at least part of a processing system for a monitoring and programming or reprogramming application and may also function to provide a feedback receiver.

FIG. 12 illustrates, by way of example and not limitation, a system 1253 that includes a stimulator 1254 and a processing system 1256, where the stimulator 1254 includes the feedback receiver 1255 with sensor(s) 1257 for sensing user action feedback. The processing system 1256 may include one or more user devices such as a phone, tablet, wearable device, or remote control, where at least some of the device(s) include a user interface 1258. Sensor(s) 1257 within the implantable or external stimulator 1254 may include, but are not limited to, accelerometers, pressure sensors, acoustic sensor(s) like a microphone, temperature sensors, and/or capacitive or resistive touch technology. Thus, the implantable or external stimulator may itself function as the feedback receiver (e.g., tap receiver). For example, the implantable stimulator may be implanted under the patient's clavicle (collarbone). The patient may tap directly over the implant or elsewhere near the implant such as the shoulder, neck, upper arm or upper thorax. The implantable stimulator may communicate data corresponding to the detected taps or other touches to an external device for use as user feedback for a monitoring and/or programming algorithm.

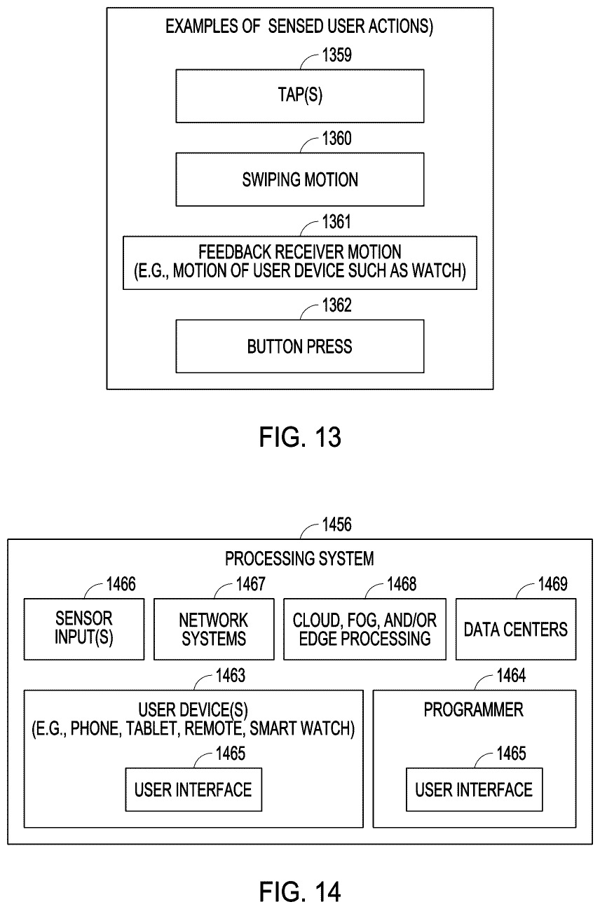

FIG. 13 illustrates, by way of example and not limitation, examples of sensed user actions that may be used to receive user feedback. These user actions may be executed by the patient in a subtle manner in public without the patient feeling like they are obviously interacting with a medical device. However, the user actions are intentional interactions with the feedback receiver. These user actions may be detected and interpreted as an intentional feedback signal from the patient using one or more of the sensors provided above. The user action capable of being sensed may include a tap or a pattern of more than one tap 1359. The user action capable of being sensed may include a swiping motion 1360 across at least a portion of a device or a region near the device. The user action capable of being sensed may include feedback receiver motion 1361. This motion refers to the motion or position of the feedback receiver itself. For example, feedback receiver such as a watch or phone may be moved, oriented or flipped in a pattern or manner to convey a feedback message. The user action capable of being sensed may include a button press 1362, which may include the press of a physical button such as a button along the side of the watch or phone or the press of a virtual button presented on a touch screen display of the feedback receiver.

FIG. 14 illustrates, by way of example and not limitation, a processing system which may be used to monitor the neurostimulation therapy, the patient condition or the therapy outcomes and to program the neurostimulator. The processing system 1456 may include one or more user device(s) 1463 and/or a programmer 1464. The processing system 1456 may be configured to implement programming and monitoring algorithm(s) using a distributed processing system where functions of the algorithms are implemented on different devices. The processing system 1456 may include user interface(s) 1465 and sensor input(s) 1466 that may provide feedback data, and this feedback data may be used to generate clinical response values 538 illustrated in FIG. 5. The processing system 1456 may include network systems 1467 and cloud, fog and/or edge processing technology 1468. Data center(s) 1469 may be used to support artificial intelligence, machine learning and analytics, which may be implemented in the programming and monitoring algorithm(s).

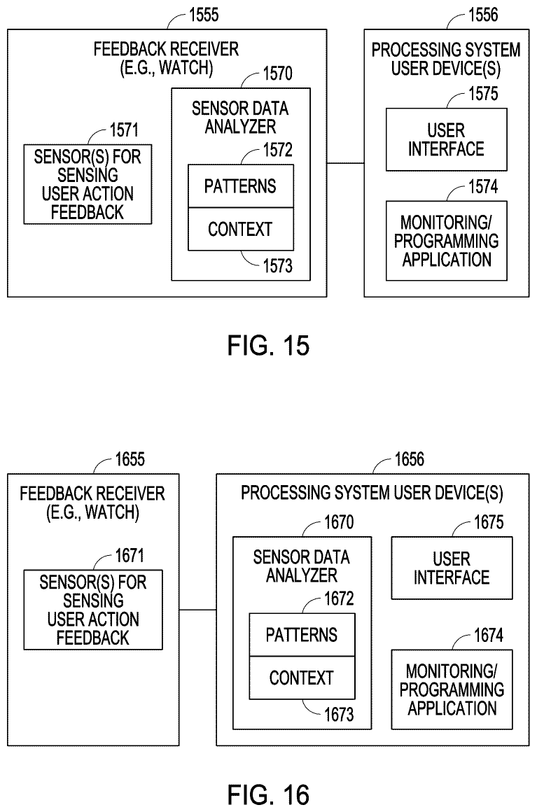

FIG. 15 illustrates, by way of example and not limitation, a feedback receiver 1555 with a sensor data analyzer 1570 configured to analyze the sensor data and communicate feedback to a monitoring and/or programming application in the processing system 1556. The illustrated feedback receiver 1555, such as but not limited to a smart watch, includes sensor(s) 1571 to detect user action and provide corresponding sensor data. The sensor data analyzer 1570 may be configured to receive and interpret the raw sensor data into feedback. For example, patterns 1572 within the sensor data may correspond to specific feedback. A single tap, a double tap, and a triple tap within a period time may correspond to different feedback messages. The rhythm between taps may further distinctions in the feedback. For example, equal durations between taps and equal intensity of the taps may correspond to one message. Other messages may be provided using different rhythms (e.g., short duration between two beats, another short duration before a subsequent beat, followed by a long duration before the last beat). Other messages may detect a rhythm where one or more of the taps are delivered with more intensity or force than other taps. The number of taps or swipes may correspond to a current rating for the therapy. Increasing number of taps or swipes may correspond to greater dissatisfaction with the current therapy. A single tap may indicate that the current therapy is great and that the patient is very satisfied. A two-tap pattern may indicate that the therapy is generally satisfactory but that improvements may able to be made. The system may query the user at a different time or more convenient time for additional information. A five-tap pattern may indicate that the therapy is not effective and that immediate intervention is desired. The monitoring/programming algorithm may revert to a previously acceptable stimulation program and may initiate further queries via the patient's user device(s) (e.g., watch or phone user interface). Some embodiments may use a combination of user actions to create feedback messages (e.g., various combinations of swipe directions/tap patterns/feedback receiver movement pattern such as but not limited to shaking back and forth or flipping or circles). Similarly, the intensity of taps may correspond to the patient's satisfaction. A soft tap may indicate the therapy is acceptable and a harder tap may indicate the therapy is not satisfactory. The system may be configured to differentiate a single finger tap from tap(s) generated using two or more fingers. Taps at different locations on the device or on different portions of the patient may create distinguishable sensor output signals. The system may be configured to create the sensor templates specific to the patient. That is, during a patient-specific setup, a patient may provide actions (e.g., a single tap with one finger at a first location and a double tap with two fingers at a second location) intended to provide different feedbacks. The corresponding sensor output is used to provide the templates that may be used to detect those specific actions in the future.

Some embodiments may use context 1573 to further interpret the feedback being provided by the raw sensor data. For example, the same user action may provide one type of feedback if it is not prompted and may provide another type of feedback if it is a response to a prompt. Different types of prompt patterns may be used to provide different queries to the patient. A single vibration may indicate a request if they are satisfied with the therapy. A double vibration may indicate a request to rate a specific symptom (e.g., tremor, rigidity, or bradykinesia) that is being treated with the therapy. Some embodiments use the same prompt (vibration and/or tone) to request a sequence of feedback. The same vibration may be used four times to provide a sequence of four prompts. The responses to the prompt sequence may indicate overall satisfaction after the first prompt, first symptom relief and/or side effect in response to the second prompt, second symptom relief and/or side effect in response to the third prompt, and third symptom relief and/or side effect in response to the fourth prompt.

The feedback may be communicated from the feedback receiver 1555 to the processing system 1556. The monitoring and/or programming application(s) 1574 implemented in the processing system 1556 may use the feedback. For example, this feedback may be used to generate clinical response values 538 illustrated in FIG. 5. The processing system 1556 may include a user interface 1575 to assist with interacting with and programming the neurostimulator.

FIG. 16 illustrates, by way of example and not limitation, a feedback receiver 1655 and a processing system 1656 with a sensor data analyzer 1670 configured to analyze the sensor data from the sensor(s) 1671 in the feedback receiver to provide feedback to a monitoring and/or programming application 1674 in the processing system 1656. The sensor data may be communicated from the feedback receiver 1655 to the processing system 1656, wherein it is analyzed to convert the sensor data into specific feedback. As provided in the discussion of FIG. 15, the analyzer may use patterns 1672 and/or context 1673 to convert the sensor data into the feedback used by the monitoring and/or programming application 1674. The processing system 1656 also includes a user interface 1675 to assist with interacting with and programming the neurostimulator.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
using a neurostimulator configured to use at least one neurostimulation program to deliver a neurostimulation therapy to treat a patient condition and provide therapy outcomes;
using a feedback receiver to receive user feedback about the neurostimulation therapy, the patient condition or the therapy outcomes, including using at least one sensor to sense at least one of user taps on or near the feedback receiver, a swiping motion across at least some of the feedback receiver, or feedback receiver movement; and
testing stimulation parameter sets using a processing system, including configuring the neurostimulator to deliver electrical energy using each of the tested stimulation parameter sets and using the user feedback to evaluate the tested stimulation parameter sets.

2. The method of claim 1, wherein the user feedback is received using at least one of a touch sensor, an accelerometer, acoustic sensor, pressure sensor for sensing at least one of user taps on the feedback receiver, motion of the feedback receiver or swipes across at least a portion of the feedback receiver.

3. The method of claim 1, wherein the user feedback is received using at least one user device.

4. The method of claim 1, wherein the user feedback is received using a body-worn device to sense at least one of user taps, the swiping motion, or the feedback receiver movement.

5. The method of claim 1, wherein the user feedback is received using a phone, a tablet or a remote control.

6. The method of claim 1, wherein the user feedback is received using the neurostimulator to sense the user taps.

7. The method of claim 1, further comprising determining the user feedback by detecting a pattern of the at least one of the user taps, the swiping motion, or the feedback receiver movement using the feedback receiver or the processing system.

8. The method of claim 1, further comprising using the feedback receiver or the processing system to determine a user-requested change in the neurostimulation therapy, user acceptance or rejection of the neurostimulation therapy, or user scoring of the neurostimulation therapy by detecting the at least one of the user taps, the swiping motion, or the feedback receiver movement.

9. The method of claim 1, wherein the feedback receiver includes a wrist-worn device and the processing system includes at least one user device configured to communicate with the wrist-worn device.

10. The method of claim 1, wherein the user feedback is received using at least one sensor in the neurostimulator, in a user device, or in a wearable device.

11. The method of claim 1, further comprising producing a user perceivable signal using the feedback receiver to prompt for the user feedback, wherein the user perceivable signal includes at least one of a vibration, a sound or a visual notification.

12. The method of claim 11, further comprising triggering the user perceivable signal based on an occurring or upcoming change in the neurostimulation therapy, the patient condition or the therapy outcomes.

13. The method of claim 11, further comprising using the user perceivable signal to prompt for a response to a question displayed on a screen.

14. The method of claim 1, further comprising collecting patient feedback in an unprompted manner using the feedback receiver, and using the collected patient feedback to provide patient driven monitoring of the neurostimulation therapy and reprogramming the neurostimulator.

15. The method of claim 1, further comprising using the user feedback to decide to change stimulation in conjunction with an optimization algorithm, trigger a change in automated patient monitoring, monitor patient outcomes and tag related stimulation data over time, trigger an optimization algorithm to begin running, or act as a trigger to change stimulation based on predefined logic.

16. A non-transitory machine-readable medium including instructions, which when executed by a machine, cause the machine to perform a method comprising:
using a feedback receiver configured to receive user feedback about a neurostimulation therapy to treat a patient condition and provide therapy outcomes, including using at least one sensor to sense at least one of user taps on or near the feedback receiver, a swiping motion across at least some of the feedback receiver, or feedback receiver movement; and
testing stimulation parameter sets using a processing system, including configuring a neurostimulator to deliver electrical energy using tested stimulation parameter sets and using the user feedback to evaluate the tested stimulation parameter sets.

17. The non-transitory machine-readable medium of claim 16, wherein the user feedback is received using at least one of a touch sensor, an accelerometer, acoustic sensor, pressure sensor for sensing at least one of user taps on the feedback receiver, motion of the feedback receiver or swipes across at least a portion of the feedback receiver.

18. The non-transitory machine-readable medium of claim 16, further comprising:
determining the user feedback by detecting a pattern of the at least one of the user taps, the swiping motion, or the feedback receiver movement using the feedback receiver or the processing system; or determining a user-requested change in the neurostimulation therapy, user acceptance of the neurostimulation therapy, or user rejection of the neurostimulation therapy by detecting the at least one of the user taps, the swiping motion, or the feedback receiver movement.

19. The non-transitory machine-readable medium of claim 16, further comprising producing a user perceivable vibration or sound using the feedback receiver to prompt for the user feedback.

20. A system, comprising:
a neurostimulator configured to use at least one neurostimulation program to deliver a neurostimulation therapy to treat a patient condition and provide therapy outcomes;
a feedback receiver configured to receive user feedback about the neurostimulation therapy, the patient condition or the therapy outcomes, wherein the feedback receiver includes at least one sensor configured to sense at least one of user taps on or near the feedback receiver, a swiping motion across at least some of the feedback receiver, or feedback receiver movement; and
a processing system configured to test stimulation parameter sets, including configuring the neurostimulator to deliver electrical energy using each of the tested stimulation parameter sets and using the user feedback to evaluate the tested stimulation parameter sets.
